(19) United States
(12) Patent Application Publication
(10) Pub. No.: US 2011/0160634 A1
(43) Pub. Date: Jun. 30, 2011

(54) CANKER SORE PATCH

(76) Inventor: Leonardo Malcovati, Lyngby (DK)

(21) Appl. No.: 12/737,263

(22) PCT Filed: Jun. 29, 2009
(86) PCT No.: PCT/DK2009/050152

§ 371 Date: Mar. 10, 2011

(30) Foreign Application Priority Data
Jun. 30, 2008 (DK) PA 2008 00909

(51) Int. Cl. A61F 13/02; B29C 43/20; A61K 9/00

(52) U.S. Cl. 602/54; 264/129

(57) ABSTRACT

An oral mucoadhesive patch used for canker sore treatments disclosed, said patch comprising a mucoadhesive layer and a protective layer, wherein the protective layer comprises pressure sensitive adhesive.

### FIELD OF THE INVENTION

The present invention relates to patches used for canker sores and a method for preparing such patches.

### BACKGROUND

Canker sores are a very common ailment, affecting an estimated 2% of any population at any instant and about 33% of all people at some points in their life. The condition, albeit not itself dangerous, is bothersome to most, especially when eating or drinking.

Canker sores are ulcers that affect mucous membranes in the mouth and usually develop on the inner cheeks, gums, lips and, occasionally, the tongue. They may occur singly as aphthous stomatitis lesions or in groups as recurrent aphthous stomatitis (RAS). The histopathology of the ulcerated lesions is similar to the one occurring under sites of acute inflammation of the skin. The canker sore is most painful during the first 3-4 days. The discomfort gradually diminishes and the sore heals in 10-14 days, usually without scarring. Before it becomes visible, the canker sore may produce a tingling or burning sensation. After 6-24 hours, the ulcer appears as a small round depression 0.5 to 9 mm in diameter, surrounded by a red area of inflammation.

Corticosteroids and other anti-inflammatory agents are used to treat the inflammation associated with canker sores. These medications, supplied in a gel, cream or paste formulation, are applied three to four times a day.

Adhesive dosage forms have been reported for buccal administration of drugs for many years. Suitable adhesive carriers include linear and partially crosslinked polymers. An ideal buccal device should be elastic, soft and able to withstand the stress caused by mouth activities. It must also possess bioadhesive properties to ensure that it is retained in the mouth for the required period of time.

The usefulness of mucosal adhesive plasters as a means of protecting these lesions and accelerating their healing is well known and several products have been available on the market for a long time.

However, mucosal adhesives do, as a general rule, swell in water and many of them are slowly soluble, which make their persistence in a wet environment, such as the human mouth, problematic.

The dissolution problem can be helped by covering one side of the plaster with a waterproof coating. Another way of solving this problem is to use rather large plasters, whose dissolution in saliva can take several hours. A third option is to improve the water-resistance of mucosal adhesives.

All the above-mentioned approaches pose problems: due to the polar, hydrophilic nature of most mucosal adhesives, standard hydrophobic coatings generally adhere to them poorly, whereas larger plasters swell to a size that usually causes considerable discomfort to the user. Finally, uncoated formulations, in order to retain mucosal adhesion, cannot guarantee resistance to food chemicals such as alcohol, oil and vinegar.

Furthermore, canker sores tend to be small in size (typically around 1 mm), and, if the plaster covering the canker sores is of comparable size, its placement may be a problem, especially in the less accessible parts of the mouth.

International Patent Application No. WO 03/059390 describes a coated mucosal adhesive; this adhesive is intended for drug delivery and therefore size and placement are not issues. On the formulation point of view, WO 03/059390 uses polyoxamers (block copolymers of ethylene and propylene glycol) in the underlying part to make it compatible with certain hydrophobic coatings. This compatibility is, however, limited, and the addition of polymers of propylene glycols can significantly decrease mucosal adhesion.

Mizrahi, B., Golenser, J., Wolnerman, J. S. and Domb, A. J., J. Pharm. Sc., 93 (12), 2004, describe mucoadhesive tablets comprising crosslinked polyacrylic acid and hydroxypropyl cellulose, absorbed with citrus oil and magnesium salt. The tablets release natural active agents for pain reduction and rapid healing of canker sores.

It has now surprisingly been found that the patch according to the invention provides an oral mucoadhesive patch, which has a long lasting adhesiveness and is easy to apply, even in small size.

### SUMMARY OF THE INVENTION

The present invention relates to an oral mucoadhesive patch comprising a mucoadhesive layer and a protective layer, wherein the protective layer comprises a pressure sensitive adhesive.

### BRIEF DESCRIPTION OF THE DRAWINGS

FIG. 1 shows the shape of a preferred embodiment of the invention.

FIG. 2 shows an illustration curve of the probe-tack test for a tackified beeswax coating.

### DETAILED DESCRIPTION OF THE PRESENT INVENTION

The present invention consists in the use of a backing, the protective layer, which is at one time compatible with the polar mucosal adhesive and is a pressure sensitive adhesive.

In one embodiment of the invention, compatibility may be achieved by using hydrophobic molecules to which small polar groups are attached, whereas the adhesive properties may be achieved by controlling the rheology of the coating, for example with the addition of adequate amounts of tackifiers and plasticisers.

The amounts of these additives are calculated in a way that the coating, under standard tack test conditions, will display a work of adhesion of 40 to 130 J/m².

In an embodiment of the invention, the protective layer is a pressure-sensitive adhesive whose strength of adhesion towards the human skin is lower than the strength of adhesion of the mucoadhesive layer towards the oral mucus tissue.

The protective layer comprising a pressure sensitive adhesive will slightly adhere to the fingertip of the person applying the patch and allow it to be positioned with great precision upon the mucosal lesion. The uncoated side of the patch will then strongly adhere to the damaged mucosal tissue, allowing it to stay in place. As the adhesion of the mucosal adhesive in wet environment is stronger than the adhesion of the pressure sensitive adhesive of the protective layer, the fingertip can be easily removed from the patch and the patch will stay in place. The wet environment in the mouth will then eliminate any residual tack from the hydrophobic coating, preventing it from adhering to food, etc. The compatibilising elements of the coating will ensure that the film will not detach from the patch as long as it remains in place.

The patch of the invention is unique in that it allows the exact placement of a patch on a canker sore.

The present invention allows the patients to use much smaller patches and therefore the discomfort associated to the treatment of canker sores is greatly reduced.

The patches according to the present invention are highly effective in the healing of both single-ulcer and RAS patients.

In an embodiment of the invention, the mucoadhesive layer comprises slightly cross-linked polyacrylic acid.

In one embodiment of the invention, the mucoadhesive layer comprises 49% to 69% by weight of slightly cross-linked polyacrylic acid plasticised with 0% to 10% (on the weight of the acid) polyethylene glycol, 14% to 40% by weight of a water-soluble modified cellulose such as hydroxypropyl cellulose, 0% to 20% by weight of chitosan and 0% to 19% by weight of a polymer that adheres to damaged mucous tissue, such as sucrose octasulphate.

In one embodiment of the invention, the slightly cross-linked polyacrylic acid is one or more selected from the group consisting of Carbopol 71G NF, Carbopol 971P NF, Carbopol 974P NF, Carbopol 934P NF and Noveon AA-1.

In an embodiment of the invention, polyacrylic acid may be replaced by polymaleates, such as International Specialty Products Gantrez® series.

According to one embodiment of the invention, the polyacrylic acid of the mucoadhesive is not mixed with a plasticiser.

According to one embodiment of the invention, hydroxypropyl cellulose may be replaced by starch, hydroxypropylmethyl cellulose or other polysaccharides such as gum tragacanth.

In another embodiment of the invention, chitosan may be replaced by galactomannan.

Optionally, the formulation may also include aromas or essential oils, such as citrus or peppermint, to improve the taste, or salts such as magnesium chloride to create an environment more favourable to the healing of the lesion.

In one embodiment of the invention, the pressure sensitive adhesive comprises a polymer selected from the group of styrenic block copolymers, ethylene-vinyl acetate copolymers and polyacrylates.

In another embodiment of the invention, the protective layer comprises 30% to 70% by weight of a styrene-ethylene-butylene-styrene block copolymer with grafted maleic anhydride, 20% to 50% by weight of a tackifier such as a modified rosin ester and 10% by weight of a plasticiser such as low-molecular-weight paraffin.

In another embodiment of the invention, the protective layer comprises wax selected from the group of beeswax and waxes of vegetal origin.

In another embodiment of the invention, the protective layer comprises beeswax, carnauba or candelilla wax tackified with 10% to 30% selected from the group consisting of colophony, modified rosin ester and hydrocarbon tackifier resin.

A protective layer based on beeswax can be applied by pressing or extruding, or it can be sprayed by heating it to temperature lower than 100° C. Furthermore, using beeswax eliminates the need for solvents.

In one embodiment of the invention, the protective layer comprises 60% to 90% wax, 0% to 10% vegetal oil and 0% to 30% selected from the group consisting of colophony, modified rosin ester and hydrocarbon tackifier resin.

According to an embodiment of the invention, the pressure sensitive adhesive formulation of the protective layer comprises shellac or zein with appropriate plasticisers (such as terpineol), or ethylcellulose plasticized with polyethylene glycol, diacetin or triacetin and tackified with rosin esters; acrylic copolymers containing acrylic acid or methacrylic acid monomer units; and block copolymers of styrene and hydrophilic acrylic monomers.

According to an embodiment of the invention, a method of preparing an oral mucoadhesive patch containing a mucoadhesive layer and a protective layer, which comprises:

(1) mixing 49% to 69% by weight of slightly cross-linked polyacrylic acid plasticised with 0% to 10% (on the weight of the acid) polyethylene glycol, 14% to 40% by weight of a water-soluble modified cellulose such as hydroxypropyl cellulose, 0% to 20% by weight of chitosan and 0% to 19% by weight of a polymer that adheres to damaged mucous tissue, such as sucrose octasulphate, in an intensive mixer and then
(2) pressing or sintering the mucoadhesive layer on standard tablet production equipment,
(3) mixing at a temperature of about 180° C. 30% to 70% by weight of a styrene-ethylene-butylene-styrene block copolymer with grafted maleic anhydride, 20% to 50% by weight of a tackifier such as a modified rosin ester, 10% by weight of a plasticiser such as low-molecular-weight paraffin, to form the mixture for the protective layer,
(4) extruding, spraying or pressing the mixture of (3) on one side of the mucoadhesive layer from (2).

According to another embodiment of the invention, a method of preparing an oral mucoadhesive patch containing a mucoadhesive layer and a protective layer, which comprises:

(1) mixing 49% to 69% by weight of slightly cross-linked polyacrylic acid plasticised with 0% to 10% (on the weight of the acid) polyethylene glycol, 14% to 40% by weight of a water-soluble modified cellulose such as hydroxypropyl cellulose, 0% to 20% by weight of chitosan, and 0% to 19% by weight of a polymer that adheres to damaged mucous tissue, such as sucrose octasulphate, in an intensive mixer and then
(2) pressing or sintering the mucoadhesive layer on standard tablet production equipment,
(3) mixing, at a temperature of about 70° C., 80% to 90% by weight of beeswax, 10% to 20% by weight of a tackifier such as a modified rosin ester, to form the mixture for the protective layer,
(4) extrude, spray or press the mixture of (3) on one side of the mucoadhesive layer from (2).

In a preferred embodiment of the invention, the protective layer may comprise 0-1% by weight of a pigment mimicking the colour of the gums.

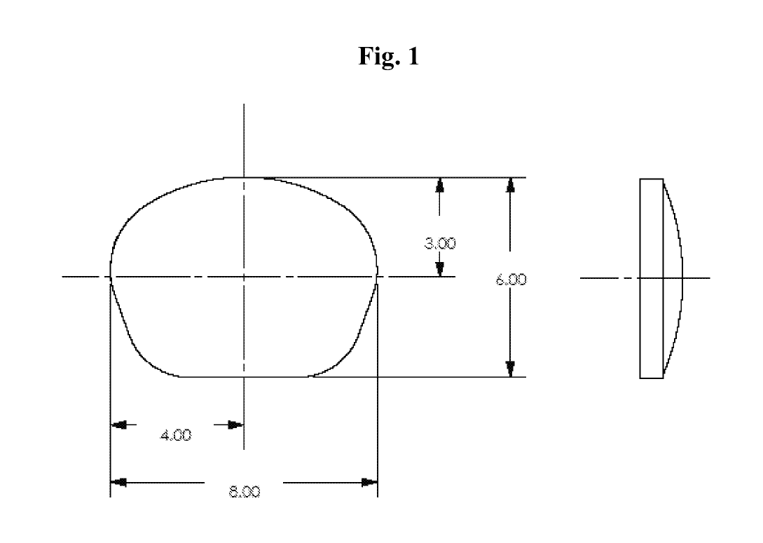

In an embodiment of the invention, the mucoadhesive patch is a mucoadhesive troche, the contour of which is described in FIG. 1, which is about 6 by 8 by 0.8 mm in size. One side of the patch is domed, in a way that the thickness is maximum (0.8 mm) in the middle and null at the edges, while the other side is completely flat.

In one embodiment of the invention, the patch is 3 to 10 mm broad in all directions and 0.2 to 1 mm thick, preferably 6 by 8 mm broad and 0.6 mm thick.

In an embodiment of the invention, the patch has one curved side with the protective layer and one substantially planar side facing the mucosal tissue.

According to one embodiment of the invention, the patch is used for the treatment of canker sore.

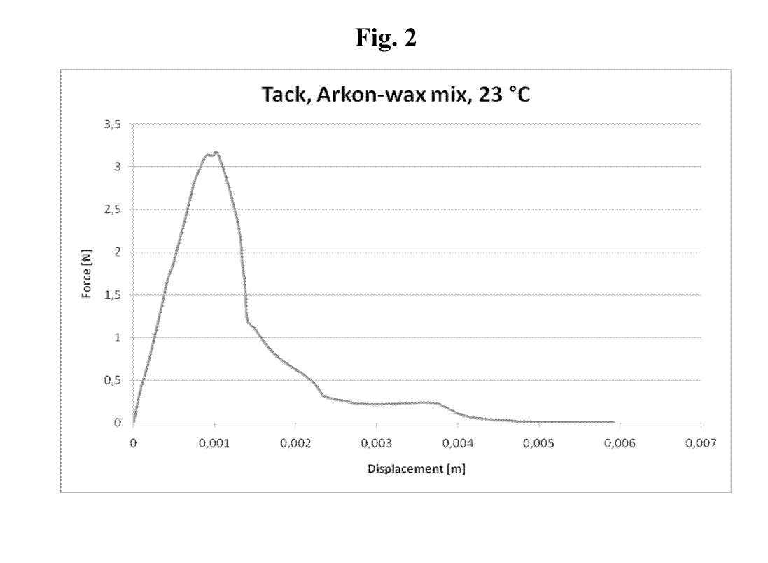

FIG. 2 shows the probe-tack curve for a tackified beeswax coating (87% beeswax, 13% hydrocarbon resin); the work of adhesion (corresponding to the area covered by the curve divided by the surface of the probe) is about 91 J/m², and the speed of the probe 10⁻⁴ m/s. The data for the curve is achieved according to a test method based on ASTM D2979.

### EXPERIMENTAL PART

### Example 1

### Materials

The following materials were used to prepare a tablet according to the invention:
Noveon AA-1, cross-linked polyacrylic acid, provided by Lubrizol
Klucel HF, hydroxypropyl cellulose, provided by Hercules
ChitoClear, chitosan, provided by Primex
S215, sucrose octasulphate, provided by Giulini Chemi
Polyethylene glycol, average molecular weight 300 Dalton, provided by Sigma Aldrich
Kraton 1924, styrene-ethylene/butylene-styrene block copolymer, provided by Kraton
Primol 352, liquid paraffin provided by ExxonMobil
Arkon P 115, a synthetic resin tackifier produced by Arakawa Chemicals
Iron oxide, provided by Sigma-Aldrich

### Methods

279 g Noveon AA-1, 47 g ChitoClear and 45 g S215 were mixed in an intensive mixer while a 5% solution of polyethylene glycol in alcohol was added dropwise to the apparatus; the total amount of polyethylene glycol constituted about 3% of the combined weight of the three materials mentioned above. After ten minutes, the mix was removed to a dryer and left there for eight hours at 50° C. The resulting granulate was mixed with 128 g Klucel HF in a powder mixer. The resulting mix was then pressed into tablets by means of a standard tablet machine.

8 g Kraton 1924, 1.5 g Arkon P115, 0.5 g Primol 352 and 0.05 g iron oxide were mixed with 100 ml cyclohexane by means of a magnetic stirrer. The resulting solution was then sprayed on the pills until a layer about 0.2 mm thick was achieved.

The covered tablets were then dried in a vacuum oven overnight in order to eliminate any remaining cyclohexane.

### Results

The backing layer was found to significantly reduce the disintegration time of the tablets in simulated saliva. The backing layer was found to slightly stick to the finger, but to be easily released from it when the other side of the tablet adhered to the gum. Finally, the product was found to relieve the pain caused by canker sores in affected people.

### Example 2

### Production Procedure with Beeswax

(1) Slightly crosslinked polyacrylic acid is mixed with anhydrous alcohol (20% to 200% on the weight of the acid) in a laminar, high-shear, low-speed mixer (e.g., planetary mixer, twin-screw mixer, etc.), by pouring the alcohol in the powdered acid over the course of a few minutes. The speed of the mixer, and the amount of alcohol are regulated so as to obtain lumps whose average size is between 0.5 cm and 1 cm across
(2) The material from (1) is dried in a vacuum oven for 2 to 8 hours at a temperature from 50 to 80° C. until at least 99.99% of the alcohol is eliminated, and the lumps have become brittle and porous
(3) The material from (3) is milled in a blade mill so that the resulting particles are around 300 μm across.
(4) The granulate from (3) is mixed in a powder mixer (inversion mixer) with chitosan, hydroxypropyl cellulose and sucralfate for about four hours in the proportions:

### Granulate (3): 49% to 60%

### Hydroxypropylcellulose: 22% to 30%

### Chitosan: 0% to 12%

### Sucralfate: 8% to 15%

The powders above will be chosen so that their average particle size is within 100 μm and 600 μm across.
(5) Beeswax (80% to 90%), Arkon P 90 (10% to 20%) are heated until they are completely melted, and mixed with a high-shear, high-speed impeller (preferably, a double crown or tooth impeller). At this point, PV fast red BNP (0.08% to 0.16% on the weight of the remaining ingredients) is added to the mix, which is then stirred until homogeneous, and cooled.
(6) The powder mix from (3) is pressed in a tablet machine to form tablets of the shape and size described in FIG. 1.
(7) The beeswax mix from (5) is pressed or sprayed on the convex side of the products (6) until a layer about 0.2 mm thick is obtained. The products are then cooled, and packed in airtight containers.

1. An oral mucoadhesive patch comprising a mucoadhesive layer and a protective layer, wherein the protective layer comprises a pressure sensitive adhesive.

2. The patch according to claim 1, wherein the pressure sensitive adhesive comprises a polymer selected from the group of styrenic block copolymers, ethylene-vinyl acetate copolymers and polyacrylates.

3. The patch according to claim 1, wherein the protective layer comprises 30% to 70% by weight of a styrene-ethylene-butylene-styrene block copolymer with grafted maleic anhydride, 20% to 50% by weight of a tackifier such as a modified rosin ester, 10% by weight of a plasticiser such as low-molecular-weight paraffin.

4. The patch according to claim 1, wherein the protective layer comprises wax selected from the group of beeswax and waxes of vegetal origin.

5. The patch according to claim 1, wherein the protective layer comprises 60% to 90% wax, 0% to 10% vegetal oil and 0% to 30% selected from the group consisting of colophony, modified rosin ester and hydrocarbon tackifier resin.

6. The patch according to claim 1, wherein the protective layer is a pressure-sensitive adhesive whose strength of adhesion towards the human skin is lower than the strength of adhesion of the mucoadhesive layer towards the oral mucus tissue.

7. The patch according to claim 1, wherein the mucoadhesive layer comprises slightly cross-linked polyacrylic acid.

8. The patch according to claim 1, wherein the mucoadhesive layer comprises 49% to 69% by weight of slightly cross-linked polyacrylic acid plasticised with 0% to 10% (on the weight of the acid) polyethylene glycol, 14% to 40% by weight of a water-soluble modified cellulose such as hydroxypropyl cellulose, 0% to 20% by weight of chitosan and 0% to 19% by weight of a product that adheres to damaged mucous tissue, such as sucrose octasulphate.

9. The patch according to claim 1, wherein the slightly cross-linked polyacrylic acid is one or more selected from the group consisting of Carbopol 71G NF, Carbopol 971P NF, Carbopol 974P NF, Carbopol 934P NF and Noveon AA-1.

10. The patch according to claim 1, wherein the patch is 3 to 10 mm broad in all directions and 0.2 to 1 mm thick, preferably 6 by 8 mm broad and 0.6 mm thick.

11. The patch according to claim 1, wherein the patch has one curved side with the protective layer and one substantially planar side facing the mucosal tissue.

12. Use of a patch according to claim 1 for the treatment of canker sore.

13. A method of preparing an oral mucoadhesive patch containing a mucoadhesive layer and a protective layer, which comprises:
(1) mixing 49% to 69% by weight of slightly cross-linked polyacrylic acid plasticised with 0% to 10% (on the weight of the acid) polyethylene glycol, 14% to 40% by weight of a water-soluble modified cellulose such as hydroxypropyl cellulose, 0% to 20% by weight of chitosan, and 0% to 19% by weight of a polymer that adheres to damaged mucous tissue, such as sucrose octasulphate,
in an intensive mixer and then
(2) pressing or sintering the mucoadhesive layer on standard tablet production equipment,
(3) mixing at a temperature of about 180° C. 30% to 70% by weight of a styrene-ethylene-butylene-styrene block copolymer with grafted maleic anhydride, 20% to 50% by weight of a tackifier such as a modified rosin ester, 10% by weight of a plasticiser such as low-molecular-weight paraffin, to form the mixture for the protective layer,
(4) extrude, spray or press the mixture of (3) on one side of the mucoadhesive layer from (2).

14. A method of preparing an oral mucoadhesive patch containing a mucoadhesive layer and a protective layer, which comprises:
(1) mixing 49% to 69% by weight of slightly cross-linked polyacrylic acid plasticised with 0% to 10% (on the weight of the acid) polyethylene glycol, 14% to 40% by weight of a water-soluble modified cellulose such as hydroxypropyl cellulose, 0% to 20% by weight of chitosan, and 0% to 19% by weight of a polymer that adheres to damaged mucous tissue, such as sucrose octasulphate,
in an intensive mixer and then
(2) pressing or sintering the mucoadhesive layer on standard tablet production equipment,
(3) mixing at a temperature of about 70° C. 80% to 90% by weight of beeswax, 10% to 20% by weight of a tackifier such as a modified rosin ester, to form the mixture for the protective layer,
(4) extrude, spray or press the mixture of (3) on one side of the mucoadhesive layer from (2).
